(19) United States
(12) Patent Application Publication
(10) Pub. No.: US 2022/0183994 A1
(43) Pub. Date: Jun. 16, 2022

(54) DELIVERY DEVICES FOR LOCALIZED DELIVERY OF ANTIMICROBIAL, ANTI-INFLAMMATORY, AND ANTIOXIDANT AGENTS

(71) Applicant: The Texas A&M University System, College Station, TX (US)

(72) Inventors: Carolyn L. Cannon, College Station, TX (US); Kush Shah, San Antonio, TX (US); Bhagath Chirra, Bryan, TX (US); Qingquan Chen, College Station, TX (US); Mansoor Khan, College Station, TX (US); Hung-Jen Wu, College Station, TX (US)

(21) Appl. No.: 17/600,837

(22) PCT Filed: Apr. 3, 2020
(86) PCT No.: PCT/US20/26715

§ 371 Date: Oct. 1, 2021

Related U.S. Application Data
(60) Provisional application No. 62/829,618, filed on Apr. 4, 2019.

(51) Int. Cl. A61K 9/70; A61K 33/38; A61K 31/197; A61K 31/375; A61K 31/4045; A61K 31/192

(52) U.S. Cl. CPC A61K 9/7007; A61K 33/38; A61K 31/192; A61K 31/375; A61K 31/4045; A61K 31/197

(57) ABSTRACT

In an embodiment, a drug delivery system and method of use thereof to treat inflammation and bacterial pathogens including multi-drug resistant pathogens, such as MDR-Pseudomonas aeruginosa, or reactive oxygen species in a subject is provided. In some embodiments, the drug delivery system includes a polymeric material, an excepient, an antioxidant agent, and an anti-inflammatory and antimicrobial agent. In some embodiments, the anti-inflammatory and antimicrobial agent is a salt of an anti-inflammatory agent and an antimicrobial agent formulated to provide a combination of antimicrobial and anti-inflammatory action from a single molecule. In some embodiments, the combined anti-inflammatory and antimicrobial agent is a silver salt of ibuprofen. In other embodiments, the combined anti-inflammatory and antimicrobial agent is unmodified, native ibuprofen.

### CROSS-REFERENCE TO RELATED APPLICATIONS

This patent application claims priority from, and incorporates by reference the entire disclosure of, U.S. Provisional Application No. 62/829,618 filed on Apr. 4, 2019.

### TECHNICAL FIELD

The present disclosure relates generally to delivery devices and more particularly, but not by way of limitation, to delivery devices for localized delivery of antimicrobial, anti-inflammatory, and antioxidant agents.

### BACKGROUND

Silver-based antimicrobials are widely used topically to treat infections associated with multi-drug resistant (MDR) pathogens. Expanding this topical use to aerosols to treat lung infections requires understanding and preventing silver toxicity in the respiratory tract. A key mechanism resulting in silver-induced toxicity is the production of reactive oxygen species (ROS). In this disclosure, the inventors have verified ROS generation in silver-treated bronchial epithelial (16HBE) cells prompting evaluation of three antioxidants, N-acetyl cysteine (NAC), ascorbic acid, and melatonin, to identify potential prophylactic agents. Among them, NAC was the only candidate that abrogated the ROS generation in response to silver exposure resulting in the rescue of these cells from silver-associated toxicity. Further, this protective effect directly translated to restoration of metabolic activity, as demonstrated by the normal levels of citric acid cycle metabolites, citrate, glutamate, aspartate, fumarate, and malate in NAC-pretreated silver-exposed cells. As a result of the normalized citric acid cycle, cells pre-incubated with NAC demonstrated significantly higher levels of adenosine triphosphate (ATP) compared with NAC-free controls. Moreover, the inventors found that this prodigious capacity of NAC to rescue silver-exposed cells was due not only to its antioxidant activity, but also to its ability to directly bind silver. Despite binding to silver, NAC did not alter the antimicrobial activity of silver.

### SUMMARY OF THE INVENTION

In an embodiment, a drug delivery platform includes a polymeric material, an excepient, an antioxidant agent, an anti-inflammatory agent, and an antimicrobial agent. In some embodiments, the anti-inflammatory agent and antimicrobial is a silver salt of ibuprofen (AgIBU) formulated to provide a combination of antimicrobial and anti-inflammatory action from a single molecule.

In another embodiment, a drug delivery platform includes a polymeric material, an excepient, an antioxidant agent, and a combined anti-inflammatory and antimicrobial agent, where the combined anti-inflammatory and antimicrobial agent is a salt of an anti-inflammatory agent and an antimicrobial agent formulated to provide a combination of antimicrobial and anti-inflammatory action from a single molecule. In some embodiments, the combined anti-inflammatory and antimicrobial agent is a silver salt of ibuprofen (AgIBU).

In an additional embodiment, a method to treat inflammation, bacterial pathogens including multi-drug resistant (MDR) pathogens, such as MDR-Pseudomonas aeruginosa, or reactive oxygen species in a subject, the method includes administering a drug delivery platform to a subject in need thereof, and where the drug delivery platform includes a polymeric material, an excepient, an antioxidant agent, an anti-inflammatory agent, and an antimicrobial agent. In some embodiments, the anti-inflammatory agent and antimicrobial is a silver salt of ibuprofen (AgIBU) formulated to provide a combination of antimicrobial and anti-inflammatory action from a single molecule.

In a further embodiment, a method to treat inflammation, bacterial pathogens including multi-drug resistant (MDR) pathogens, such as MDR-Pseudomonas aeruginosa, or reactive oxygen species in a subject, the method includes administering a drug delivery platform to a subject in need thereof, and where the drug delivery platform includes a polymeric material, an excepient, an antioxidant agent, and a combined anti-inflammatory and antimicrobial agent, where the combined anti-inflammatory and antimicrobial agent is a salt of an anti-inflammatory agent and an antimicrobial agent formulated to provide a combination of antimicrobial and anti-inflammatory action from a single molecule. In some embodiments, the combined anti-inflammatory and antimicrobial agent is a silver salt of ibuprofen (AgIBU).

In an embodiment, a drug delivery liposome including a polymeric material, a glycan, an excepient, an antioxidant agent, and a combined anti-inflammatory and antimicrobial agent, where the combined anti-inflammatory and antimicrobial agent is a salt of an anti-inflammatory agent and an antimicrobial agent formulated to provide a combination of antimicrobial and anti-inflammatory action from a single molecule. In some embodiments, the combined anti-inflammatory and antimicrobial agent is a silver salt of ibuprofen (AgIBU). In other embodiments, the combined anti-inflammatory and antimicrobial agent is unmodified, native ibuprofen.

In another embodiment, a method to treat inflammation and bacterial pathogens including multi-drug resistant (MDR) pathogens, such as MDR-Pseudomonas aeruginosa, or reactive oxygen species in a subject is provided, where the method includes administering a drug delivery liposome to a subject in need thereof, and where the drug delivery liposome includes a polymeric material, a glycan, an excepient, an antioxidant agent, and a combined anti-inflammatory and antimicrobial agent, where the combined anti-inflammatory and antimicrobial agent is a salt of an anti-inflammatory agent and an antimicrobial agent formulated to provide a combination of antimicrobial and anti-inflammatory action from a single molecule. In some embodiments, the combined anti-inflammatory and antimicrobial agent is a silver salt of ibuprofen (AgIBU). In other embodiments, the combined anti-inflammatory and antimicrobial agent is unmodified, native ibuprofen.

### BRIEF DESCRIPTION OF THE DRAWINGS

A more complete understanding of the subject matter of the present disclosure may be obtained by reference to the following Detailed Description when taken in conjunction with the accompanying Drawings wherein:

FIG. 1A and FIG. 1B show reactive oxygen species and superoxide levels in human bronchial epithelial (16HBE) cells upon exposure to silver acetate (AgAc) for 8 h (FIG. 1A) and 24 h (FIG. 1B);

FIG. 2 shows a comparison of the antioxidant activity of N-acetyl cysteine (NAC), ascorbic acid (AA), and melatonin at 10 mM concentration;

FIG. 3A, FIG. 3B, FIG. 3C and FIG. 3D show reactive oxygen species (FIGS. 3A and 3B) and superoxide (FIGS. 3C and FIGS. 3D) levels in human bronchial epithelial (16HBE) cells upon pre-incubation with 0 or 10 mM NAC followed by a 8-hour (FIG. 3A and FIG. 3C) or 24-hour (FIG. 3B and FIG. 3D) exposure to silver acetate at various concentrations demonstrating the capacity of NAC to abrogate ROS and superoxide production. **: p<0.01 and ****: p<0.0001;

FIG. 4 shows ATP production in human bronchial epithelial (16HBE) cells pre-incubated with 0 or 10 mM N-acetyl cysteine (NAC) followed by exposure to silver acetate for 8 h demonstrating normalization of ATP production after silver exposure by NAC pre-incubation. ****: p<0.0001;

FIG. 5 shows scanning electron micrographs of (a) drug-free and (b) AgIBU loaded poly(caprolactone) electro-spun scaffolds;

FIG. 6 shows hetero-multivalent liposome targeting PA within biofilms. Retention of fluorescent liposomes on PA (PAO1) was quantified by normalized fluorescence intensity per colony forming unit (CFU). Control: no ligand; Single Ligand: 10 mol % Gb3 or 10 mol % LacCer; Mixed Ligands: 5 mol % Gb3+5 mol % LacCer. The error bars are standard deviation (n=3);

FIG. 7 shows targeted liposomes (5% Gb3+5% LacCer) colocalizing with bacteria in the blood, spleen, liver, thigh, heart, and lungs within 2h post-injection, in vivo; and

FIG. 8 illustrates the mechanism of hetero-multivalent targeting.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Although silver is a potent, broad-spectrum antibiotic, silver-induced toxicity, primarily due to generation of ROS, remains a concern limiting its use beyond treatment of wounds. NAC has been widely used as an antioxidant to rescue eukaryotic cells from metal-associated toxicity. The capacity of NAC to abrogate silver toxicity in a human bronchial epithelial cell line (16HBE) was evaluated as a step towards expanding the use of silver-based antimicrobials to treat lung infections. It was found that NAC pre-incubation resurrects a healthy metabolic state in bronchial epithelial cells exposed to silver ions via a combination of its antioxidant and metal-binding properties. Finally, this ability of NAC to rescue silver-exposed eukaryotic cells does not alter the antimicrobial activity of silver. Thus, a silver-NAC combination holds tremendous potential as a future, non-toxic antimicrobial agent.

Silver is a mainstay therapeutic strategy for prophylaxis, as well as eradication, of established infections in wound and burn patients. This wide-spread use of silver stems from its broad-spectrum antimicrobial activity and multiple mechanisms of action including disruption of bacterial cell walls, and DNA condensation. These multiple mechanisms impart potent biocidal activity against several bacterial pathogens including multi-drug resistant (MDR) Pseudomonas aeruginosa, Staphylococcus aureus, Escherichia coli, as well as fungus, mold, and yeast. The ability of silver to target multiple pathways also lowers the propensity of resistance acquisition by microbes, which is commonly observed amongst antibiotics with single targets. Only a few cases of silver resistance have been reported. Thus, silver has been incorporated into, or used as a coating in, over 400 medical and consumer products including wound dressings, catheters and endotracheal tubes, bone cement, socks, and disinfectants. In addition to its antimicrobial activity, silver has also garnered attention as a potential anticancer agent. Despite this tremendous potential, stability and toxicity are two major limitations that hamper the use of silver as a therapeutic on a larger scale.

The oligodynamic effects of silver are limited to its ionic form (+1 oxidation state; Ag⁺), which has a high affinity for chloride ions, as well as thiol functionalized substrates and proteins. Interaction with these functional groups often results in deactivation of the silver ion and loss of biological activity. The inventors have developed a library of silver-based antimicrobials, silver carbene complexes (SCCs), with enhanced stability over conventional silver salts. These molecules have demonstrated superior antimicrobial activity against clinically relevant MDR pathogens including Pseudomonas aeruginosa, Burkholderia cepacia complex species, Staphylococcus aureus, Klebsiella pneumoniae, and Acinetobacter baumannii, both in vitro and in vivo. These compounds also demonstrate potent antimicrobial activity against biodefense pathogens Bacillus anthracia and Yersinia pestis. Further, polymeric nanoparticles loaded with these SCCs demonstrate superior in vivo antimicrobial activity over parent molecules. These devices offer sustained release of the therapeutic at the infection site and protect the silver ions from deactivation. As a result, mice treated with nanoparticles result in increased survival and superior eradication of bacterial burden with fewer and lower doses compared with the parent SCCs, in an acute pneumonia model. Thus, development of novel molecules and delivery devices have addressed the stability concerns and significantly improved the efficacy of silver, opening up new avenues for the use of silver beyond topical therapy.

Toxicity of silver has always been a controversial topic. Several publications report silver to be non-toxic, with argyria, a rare and irreversible pigmentation of the skin caused by silver deposition, as the only reported side-effect. On the other hand, several reports have demonstrated toxic side effects of silver in eukaryotic cells; claims that are underscored by the anticancer activity of silver. While silver toxicity and chemotherapeutic activity have been reported, little is known about the molecular mechanisms that contribute to silver toxicity. Recently, several reports have focused on identifying the mechanisms that contribute to toxicity towards eukaryotic cells, and are also responsible for the anticancer activity of silver nanoparticles. These reports largely focus on the effect of size and surface coatings on toxicity of metallic silver nanoparticles. In general, pure silver nanoparticles demonstrate lower toxicity to eukaryotic cells compared with ionic silver at comparable concentrations, likely caused by the gradual release of ionic silver from the nanoparticles upon surface oxidation or dissolution. A direct correlation between dissolution of nanoparticles and subsequent release of silver ions to toxicity towards eukaryotic cells has been established. While the individual toxicity caused by nanoparticles and silver ions has yet to be discerned, generation of reactive oxygen species (ROS) has been implicated as a key underlying mechanism of toxicity. ROS and the complementary cellular antioxidant defense system are part of a complex cellular milieu that plays critical roles in several biochemical processes. Silver disrupts the mitochondrial respiratory chain resulting in overproduction of ROS, leading to oxidative stress, ultimately causing lipid peroxidation and protein denaturation, interruption of ATP production, DNA damage, and induction of apoptosis. Thus, ROS overproduction is one of the primary mechanisms responsible for inhibition of cell proliferation and induction of cell death in cells exposed to silver. NAC has been employed as an antioxidant to abrogate ROS generation and alleviate toxicity of silver towards eukaryotic cells. However, the effects of anti-oxidants such as NAC on the overall cellular health and cell metabolism are not well known.

Therefore, it is desirable to develop non-toxic therapeutic strategies for eradication of multi-drug resistant bacterial pathogens, particularly pathogens responsible for lung infections. Embodiments of the invention are directed towards methods and compositions that ameliorate the toxicity of silver antimicrobial compounds when administered to humans for therapeutic purposes.

An embodiment of the claimed invention is directed to evaluating the impact of silver-based antimicrobial compounds on host cellular metabolism. With an eye on developing silver-based antimicrobials to treat lung infections, the toxicity of silver in a human bronchial epithelial cell line (16HBE) was evaluated.

Another embodiment of the claimed invention is directed toward determining the effect of antioxidants on ameliorating the toxicity of silver-based antimicrobial compounds. Specifically, three antioxidants, ascorbic acid (vitamin C), melatonin, and NAC, were evaluated with respect to their effects on cell viability. NAC was shown to the only antioxidant that caused a reduction of silver toxicity. Pre-incubation with NAC rescued the cells from switching exclusively to anaerobic respiration and maintained ATP production via the electron transport chain in the mitochondria. NAC pre-incubation suppressed ROS generation and maintained metabolic activity of the cell by sequestering silver ions to abrogate silver toxicity.

Reference will now be made to more specific embodiments of the present disclosure and data that provides support for such embodiments. However, it should be noted that the disclosure below is for illustrative purposes only and is not intended to limit the scope of the claimed subject matter in any way.

Reagents. Silver acetate, Dulbecco's Modified Eagle's Medium (DMEM) powder (without glucose, phenol red, L-glutamine, sodium pyruvate, and sodium bicarbonate), D-glucose, L-glutamine, sodium bicarbonate (NaHCO₃), HEPES buffer, penicillin-streptomycin (100× stock), trypsin-EDTA solution, sodium hydroxide (NaOH, 1N), methanol, Minimum Essential Medium (MEM) with Earle's Balanced Salts and non-essential amino acids, fetal bovine serum (FBS), and N-acetyl cysteine (NAC) were obtained from Sigma-Aldrich Corporation (St. Louis, Mo.). Uniformly labeled [U¹³C] glucose was obtained from Cambridge Isotope Laboratories, Inc. (Andover, Mass.). Opti-MEM (without phenol red), ALAMARBLUE® Cell Viability Kit (Cat #DAL1100), ATP Determination Kit (Cat #A22066), and Phosphate Buffered Saline (PBS) solution (10×) were obtained from Thermo Fisher Scientific, Inc. (Waltham, Mass.). Total Antioxidant Capacity Assay Kit (Cat #ab65329), Cellular ROS/Superoxide Detection Assay Kit (Cat #ab139476), GSH/GSSG Ratio Detection Assay Kit II (Cat #ab205811), Deproteinizing Sample Kit (Cat #ab204708), and Mammalian Cell Lysis Buffer 5× (Cat #ab179835) were purchased from Abcam (Cambridge, Mass.). Tissue culture flasks, tissue culture dishes (Φ=60 mm), 24-well plates, 96-well plates, Tryptic soy agar (TSA) plates, and Mueller-Hinton (MH) broth were obtained from Becton Dickinson and Company (Franklin Lakes, N.J.), respectively. Distilled deionized water (DH₂O) was obtained from a Milli-Q biocel system (Millipore, Billerica, Mass.) and sterilized in an autoclave. All the above chemicals were used without further purification.

Cell culture. Human bronchial epithelial cell line (16HBE14o-) generously provided by Dr. D. Gruenert (University of California, San Francisco, Calif.) are a human bronchial epithelial cell line transformed with SV40 large T-antigen using the replication-defective pSVori plasmid. 16HBEs were used between passages of 20 and 40 for all experiments. 16HBE cells were cultured in Minimum Essential Medium (MEM) with Earle's Balanced Salts and non-essential amino acids supplemented with 10% fetal bovine serum (FBS), 1% L-glutamine, and 1% penicillin-streptomycin (P/S) solution at 37° C. in an incubator (5% CO₂, 100% RH), unless otherwise noted. When the cells reached 90-95% confluency, they were harvested by trypsinizing and sub-cultured.

Silver induction of reactive oxygen species (ROS) and superoxide. Cellular ROS and superoxide levels were measured in 16HBE cells using a Cellular ROS/Superoxide Detection Assay Kit according to manufacturer's recommended protocol. Briefly, cells were seeded at a density of 25,000 cells/well in a black wall/clear bottom 96-well plate and incubated for 24 h as described above. Next, the feeding media was aspirated and cells were incubated with fresh media supplemented with or without 10 mM NAC for 2 h. Finally, the NAC solution was removed and cells were incubated with 100 μL of 0, 10, 20, 50, or 100 μg/mL silver acetate containing 1× ROS/Superoxide detection mix. Upon staining, the fluorescence signal from the two fluorescent dyes, green signal from ROS detection probe (Ex/Em=490/525 nm) and orange signal from superoxide detection probe (Ex/Em=550/620 nm), were quantified using a BioTek Instruments Cytation 5 Multimode Reader at 0, 4, 6, 8, and 24 h. The fluorescence signal was normalized to the drug free controls (0 mM NAC+0 μg/mL silver acetate). All experiments were performed with 6 technical replicates and a minimum of 2 biological replicates.

Activity of antioxidants. Antioxidant activity of NAC, ascorbic acid, and melatonin was measured using a Total Antioxidant Capacity Assay Kit according to manufacturer's recommended protocol. A standard curve correlating the Trolox concentration to the antioxidant capacity was generated according to manufacturer's protocol. NAC, ascorbic acid, and melatonin were dissolved at 10mM concentration in distilled—deionized water (d. d. water) and serially diluted. All experimental and standard solutions were protected from light, incubated with colorimetric Cu⁺² probe for 1.5 h with constant shaking and absorbance was measured at 570 nm using a BioTek Cytation 5 Multimode Reader. The antioxidant capacity of test solutions, NAC, ascorbic acid, and melatonin were then correlated to the standard curve and presented as a function of the final Trolox concentration. Next, the antioxidant capacity of NAC, ascorbic acid, and melatonin pre-incubated 16HBE cells was also measured. Two million 16HBE cells were seeded into each well of a 12-well plate and incubated overnight as described above. The feeding media was then replaced with fresh feeding media or media containing 10 mM NAC, ascorbic acid, or melatonin. After a 2 h incubation with the antioxidants, cells were washed with cold PBS, re-suspended in 100 μL d. d. water, homogenized by pipetting, and incubated on ice for 10 min. Finally, the insoluble cell debris was removed by centrifugation and the supernatant analyzed as described above to determine the total antioxidant capacity. All experiments were performed with 4 technical replicates and two biological replicates.

Abrogation of silver acetate toxicity through pre-incubation with antioxidants. Toxicity of silver acetate with or without pre-incubation with antioxidants was assessed on 16HBE cells using an ALAMARBLUE® Cell Viability Assay according to manufacturer's recommended protocol. Cells were seeded at a density of 25,000 cells/well in a 96-well plate and incubated overnight as described above. At 24 h, media was aspirated, and cells were pre-incubated with 80 μL of 0, 0.01, 0.1, 1.0, 2.5, 5, 7.5, and 10 mM concentrations of NAC, ascorbic acid, and melatonin for 2 h. Next, the antioxidant supplemented media was replaced with 100 μL feeding media containing 0, 10, 20, 30, 40, 50, 75, and 100 μg/mL silver acetate. ALAMARBLUE® test reagent was added to each well, and the plates were incubated as described above. At 8-hour and 24-hour timepoints, absorbance was measured at 570 and 600 nm, normalized to media only controls, and analyzed per manufacturer's instructions. All experiments were performed with 6 technical replicates and 3 biological replicates. These results were verified using a CYQUANT® Cell Proliferation Assay Kit.

Glutathione concentrations after pretreatment with NAC. Glutathione levels, in 16HBE cells pre-incubated with NAC, were determined using a GSH/GSSG Ratio Detection Assay Kit II according to manufacturer's recommended protocol. Five million 16HBE cells were seeded in each well of a 6-well plate as described above. At 24 h, the feeding media was replaced with fresh feeding media supplemented with or without 10 mM NAC and incubated for 2 h. Next, cells were incubated with 0, 10, and 100 μg/mL silver acetate for 1 h and glutathione levels measured. Briefly, cells were washed with cold PBS, re-suspended in 300 μL ice cold Mammalian Cell Lysis Buffer and homogenized by pipetting. The cell lysate was then centrifuged to remove the cell debris and the supernatant was carefully collected and deproteinized using a Deproteinizing Sample Kit. The deproteinized samples were then diluted using lysis buffer, mixed with glutathione (GSH) and total glutathione (TGAM or GSH+GSSG) assay probes, incubated for 60 minutes protected from light, and fluorescence signal measured at Ex/Em=490/520 nm using a BioTek Cytation 5 Multimode Reader. The fluorescence signal from the experimental values were then correlated to the glutathione (GSH and GSH+GSSG) standard curves generated to determine the intracellular glutathione concentrations. Experiments were performed with 4 technical replicates and 3 biological replicates.

Analysis of total metabolite pool size and metabolite labeling patterns using Gas Chromatography-Mass Spectroscopy. 16HBE cells were seeded at a density of 250,000 cells per dish in a 60 mm cell culture dish and incubated until they reached 90% confluency. During this period, feeding media was replaced every 48 h. Once confluent, the media was aspirated, cells were washed with 1× PBS, and incubated with 2 mL 0 or 10 mM NAC for 2 h. Next, the NAC supplemented media was replaced with 2 mL 4 mM GLN-10mM D-[U¹³C]-GLC medium containing 0, 10, 20, 30, 40, 50, 75, and 100 μg/mL silver acetate. At 8 h, the feeding medium from each plate was collected, centrifuged at 1000 rpm for 5 min to remove any cell debris, and frozen at −80° C., until further analysis. The cells were washed twice with 1× PBS, re-suspended by gentle scraping in 1 mL chilled 50% methanol solution, cell lysate collected in centrifuge tubes, flash frozen using liquid nitrogen, and stored at −80° C. till analysis.

The supernatant obtained from cells pre-treated with or without NAC and exposed to various concentrations of silver acetate in 4 mM GLN-10 mM D-[U¹³C]-GLC medium (all time points) was thawed and analyzed for concentrations of glucose and lactate using a BioProfile BASIC Analyzer (Nova Biomedical, Waltham, Mass.). MEM and stock solution of 4 mM GLN-10 mM D-[U¹³C]-GLC medium treated in an identical manner were used as controls.

Cell suspensions frozen in 50% methanol were thawed and subjected to three additional freeze-thaw cycles using liquid nitrogen and a water bath. Subsequently, the cell suspensions were centrifuged at 14,000 rpm for 10 min to remove cell debris, and the supernatants were transferred to individually labeled glass drying tubes. 10 μl of an internal standard (50 nmols of sodium 2-oxobutyrate) was added to each tube at this time, and the samples were air-dried on a heat block. The dried samples were derivatized by addition of 100 μl of Tri-sil HTP reagent (Thermo Scientific) to each tube, capping the tube, vortexing the samples, and placing them on the heat block for an additional 30 min. The derivatized samples were transferred to auto-injector vials and analyzed using gas chromatography-mass spectroscopy (GC-MS; Agilent Technologies, Santa Clara, Calif.). Separately, the cell pellets with residual cell lysate was collected, contents thoroughly mixed with 200 μL 0.1 N sodium hydroxide, and heated to 100° C. to extract and solubilize the proteins. The samples were cooled and analyzed using a standard BCA assay to quantify the protein content. All metabolite concentrations determined using the BioProfile Basic-4 analyzer (NOVA) and GC-MS were normalized with the protein content.

Determination of ATP content. ATP production by 16HBE cells with and without pre-incubation with NAC followed by incubation with silver acetate was determined using an ATP determination kit using manufacturer's recommended protocol. 50,000 16HBE cells were seeded in each well of a 96-well plate and the cells were allowed to attach. At 24 h, media was aspirated, and cells were pre-incubated with 80 μL of 0 or 10 mM concentrations of NAC for 2 h. Next, the antioxidant supplemented media was replaced with 100 μL feeding media containing 0, 10, 20, 30, 40, 50, 75, and 100 μg/mL silver acetate. Following an 8 h incubation with silver acetate, the media was aspirated, cells were washed with 100 μL PBS, and incubated with 100 μL lysis buffer for 15 min. Finally, the cell lysate was collected and the ATP concentration determined and correlated to an established standard curve. Briefly, a standard reaction mixture consisting of molecular grade water, reaction buffer, Dithiothreitol (DTT) solution, D-luciferin, and firefly luciferase at manufacturer recommended concentrations was prepared. Next, 10 μL of standard ATP solution or cell lysate was mixed with 90 μL standard reaction mixture in a 96-well white bottom plate and luminescence was measured at 560 nm. Background luminescence was subtracted from all readings and the data were normalized to drug free controls.

Antimicrobial activity of silver. Antimicrobial activity of silver was evaluated against laboratory and clinical isolates of Pseudomonas aeruginosa (PA O1, PA M57-15, PA HP3, and PA14) as well as methicillin-resistant Staphylococcus aureus (MRSA; USA 300, MRSA 0606, MRSA 0638, and MRSA 0646), with or without pre-incubation with NAC. Frozen stocks of bacteria were struck onto TSA plates and allowed to grow for 18-24 h at 37° C. A single colony was used to inoculate 5 mL MH broth and grown to an OD₆₅₀=0.40 at 37° C. on an orbital shaker. Next, the bacteria were centrifuged at 2500 rpm for 15 m at 4° C., supernatant aspirated, and bacterial pellets were re-suspended in 2 mL MH broth supplemented with 0 or 10 mM NAC. Bacterial suspension was then incubated at 37° C. with orbital shaking for 2 h, centrifuged again to remove the NAC solution, and re-suspended in NAC free MH broth to OD₆₅₀=0.4. Finally, minimum inhibitory concentrations (MIC) against silver acetate were determined using standard Clinical and Laboratory Institute (CLSI) broth-microdilution method. Briefly, bacterial suspension at a concentration of 5E5 colony forming units (CFU) per milliliter was incubated with a silver acetate at a final concentration of 0.13, 0.25, 0.5, 1, 2, 4, 8, 16, and 32 μg/mL silver acetate at 37° C. for 18-24 h, under static conditions. The MIC was determined as the lowest concentration resulting in no bacterial growth upon visual inspection. All experiments were performed in triplicate.

Statistics. All data were analyzed using GraphPad Prism 7 (GraphPad Software, Inc., La Jolla, Calif.). A two-way analysis of variance (ANOVA) followed by a post hoc Sidak's or Tukey's test with multiple comparisons between means at each concentration of silver acetate was used to determine the significant difference. Additionally, non-linear regression was used to deduce the lethal dose at median cell viability (LD₅₀) for cell viability assays. A p≤0.05 was considered significantly different.

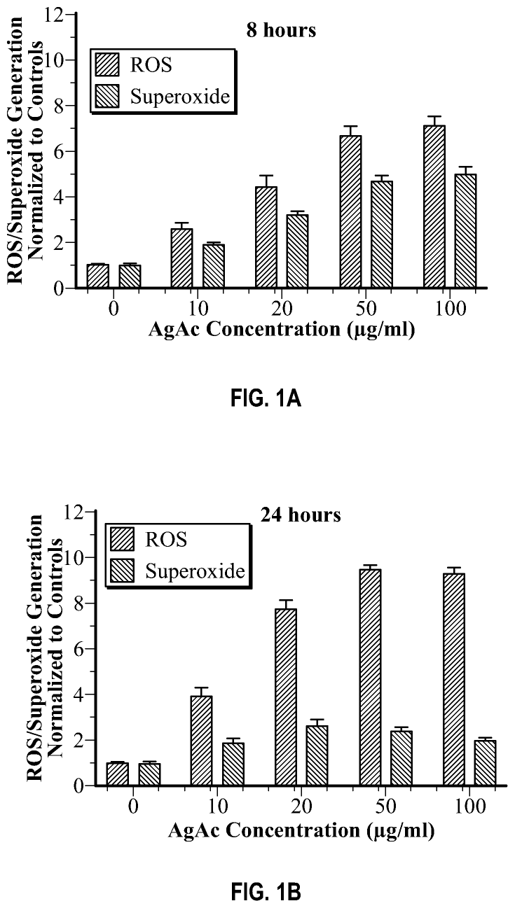

Silver induction of reactive oxygen species (ROS) and superoxide. Several publications have demonstrated ROS generation by eukaryotic cells after exposure to silver. Thus, the inventors verified the observation that silver acetate induces reactive oxygen species and superoxide ions in a human bronchial cell line, 16HBE. The inventor's results demonstrate a significantly higher amount of ROS and superoxide ions within cells that are incubated with silver acetate, at 8 hours (FIG. 1A) and 24 hours (FIG. 1B), compared with cells that are not exposed to any silver.

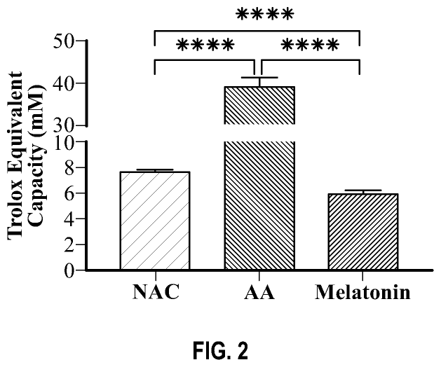

Activity of antioxidants. Quantifying the antioxidant activity of NAC, ascorbic acid, and melatonin was performed. The antioxidant activity of these molecules was compared with a standard antioxidant, trolox, and is presented as trolox equivalent activity (FIG. 2). These results demonstrate ascorbic acid to be the most powerful antioxidant with 40-fold trolox equivalent, while NAC and melatonin demonstrate ˜5-7 fold higher antioxidant capacity compared with trolox. Thus, all three anti-oxidants evaluated demonstrate potent anti-oxidant activity. Ascorbic acid demonstrates significantly higher antioxidant activity compared with melatonin and NAC.

Abrogation of silver acetate toxicity through pre-incubation with antioxidants. The effect of antioxidant pre-incubation on silver acetate toxicity towards 16HBE cells is shown in Table 1. Cells pre-incubated with NAC demonstrate significantly higher survival upon exposure to silver acetate.

TABLE 1
16HBE LD₅₀ values (μg/mL) upon exposure to silver
acetate after pre-incubation with select anti-oxidants
at 0, 2.5, 5, 7.5, and 10 mM concentrations.

| | | | |
|---|---|---|---|
| Antioxidant | NAC | Ascorbic Acid | Melatonin |

| | | | | |
|---|---|---|---|---|
| 0 | mM | 7.8 | 6.2 | 7.4 |
| 2.5 | mM | 26.6 | 5.3 | 6.2 |
| 5 | mM | 35.8 | 4.7 | 6.7 |
| 7.5 | mM | 63.5 | 5.0 | 6.5 |
| 10 | mM | 60.0 | 7.4 | 7.1 |

Pre-incubation with 7.5 and 10 mM ascorbic acid followed by up to 100 μg/mL silver acetate exposure also results in significantly (p<0.05) higher cell survival. Despite the significance, the LD₅₀ values upon pre-incubation with ascorbic acid does not appreciably change. In addition, cells exposed to 50 μg/mL silver acetate after 10 mM NAC pre-incubation result in 86% survival (p<0.0001) compared with 22% survival upon pre-incubation with 10 mM ascorbic acid. Finally, melatonin pre-incubation does not alter the toxicity of silver acetate as demonstrated by the cell survival and LD₅₀ values. Thus, of the three anti-oxidants evaluated, only NAC rescues the cells from silver acetate toxicity. Thus, NAC was chosen as the antioxidant of interest for further investigation.

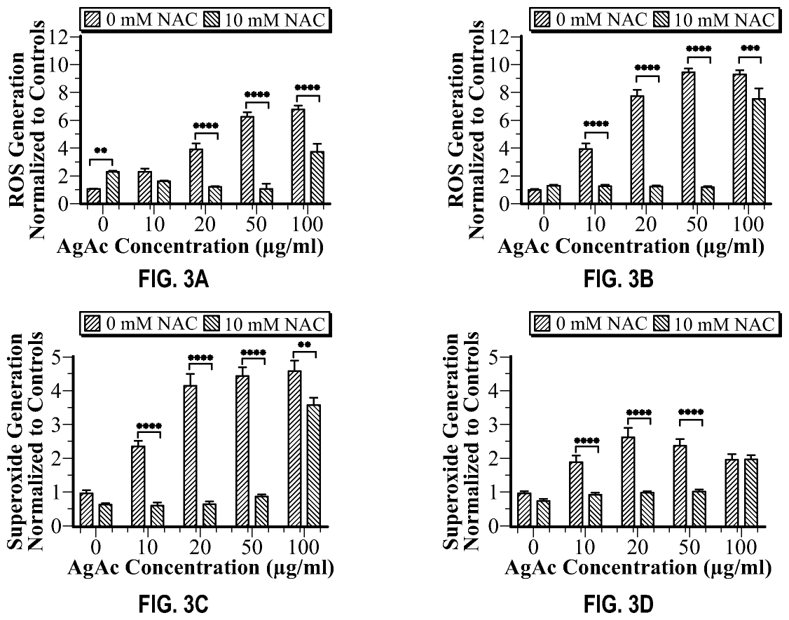

Silver induction of reactive oxygen species (ROS) and superoxide. FIGS. 3A to 3D illustrate the effect of NAC on silver acetate induced reactive oxygen species and superoxide ions. Pre-incubation with NAC suppresses the levels of ROS and superoxide seen after incubation with silver acetate for 8 and 24 hours. Specifically, cells pre-incubated with 10 mM NAC, upon exposure to silver acetate concentrations higher than 20 μg/mL, show significantly (p≤0.001) lower ROS levels at 8 and 24 hours. Similarly, when cells are pre-incubated with 10 mM NAC, superoxide levels are significantly (p≤0.001) lower at 8 and 24 hours after incubation with 10, 20, and 50 μg/mL silver acetate. Cells incubated with 100 μg/mL silver acetate show significantly (p≤0.01) lower superoxide levels at 8 hours when pre-incubated with NAC, but not at 24 hours. Surprisingly, NAC pre-incubation initially induces ROS, which subsides after 8 hours, but has no effect on superoxide levels.

NAC is a known precursor of glutathione and the effect of NAC pre-incubation on both oxidized and reduced glutathione concentrations showed the absence of correlation between ROS generation and oxidation of glutathione, after silver incubation.

Analysis of total metabolite pool size and metabolite labeling patterns using gas chromatography-mass spectroscopy. Disruption of the mitochondrial electron transport chain has been linked to the ROS overproduction and cell death upon exposure to silver ions. To further explore the metabolic effects of silver-induced ROS production, the inventors evaluated glucose consumption and its metabolism through the glycolysis pathway. Glucose consumption and lactate production, the end product of glycolysis, was determined. No significant difference is observed in glucose consumption and lactate production between cells that were pre-treated with 0 and 10 mM NAC. Thus, incubation with NAC does not significantly alter glucose consumption and lactate production. As expected, treatment with increasing concentrations of silver acetate resulted in reduced glucose consumption and lactate production. The effect of NAC incubation on the oxidation of glucose-derived carbon in the TCA cycle was determined. Cells that are not incubated with NAC demonstrate significantly (p≤0.05) lower levels of citrate, glutamate, aspartate, fumarate, and malate, but not lactate, in comparison with NAC incubated cells. In particular, citrate, glutamate, fumarate, and malate levels are significantly (p≤0.05) higher for NAC pre-incubated cells after exposure to 30, 40, 50, and 75 μg/mL silver acetate. Aspartate levels are significantly (p≤0.05) higher for NAC pre-incubated cells upon exposure to 50 and 75 μg/mL silver acetate. Finally, lactate levels were significantly higher for cells pre-incubated with NAC and exposed to 50 μg/mL silver acetate only. The lactate levels determined using GC-MS also follow similar trends compared with the levels determined using the bioprofile analyzer. In addition, pre-incubation with NAC does not appreciably alter the labeling patterns of key metabolites. Thus, these results demonstrate that silver acetate incubation results in bypass of the TCA cycle and the consumed glucose is converted to pyruvate via glycolysis, reduced to lactate, and secreted. However, upon pre-incubation with NAC, mitochondrial stress is ameliorated, as evident by the significantly higher levels of TCA cycle intermediates.

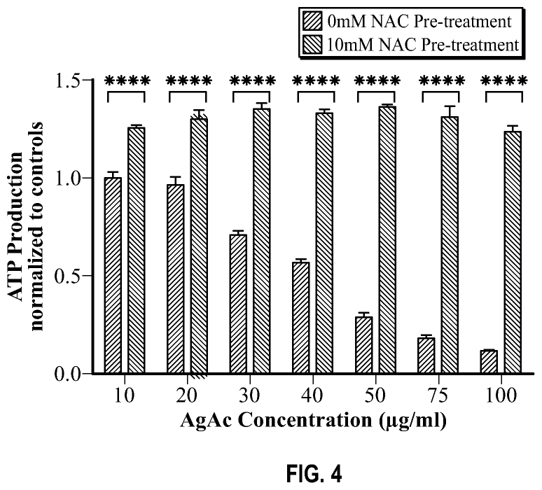

Determination of ATP content. NAC pre-incubation rescues the cells from the detrimental effects of silver disruption of the TCA cycle. Next, the downstream effect of TCA cycle salvage by NAC was measured in terms of ATP production to demonstrate the rescue of aerobic respiration in these cells (FIG. 4). Cells pre-incubated with 10 mM NAC demonstrate significantly higher ATP production upon exposure to silver compared with cells exposed to silver alone. Thus, NAC pre-incubation rescues cells from disruption of the TCA cycle and electron transport chain to maintain ATP production at a comparable rate to the control group.

Antimicrobial activity of silver with or without NAC pretreatment. Antimicrobial activity of silver acetate with or without pre-incubation with NAC was measured using a standard CLSI broth-microdilution method. The minimum inhibitory concentration (MIC) of silver acetate does not change when the bacteria are pre-incubated with 0 or 10 mM NAC, demonstrating the selectivity of NAC to rescue eukaryotic cells without altering its antimicrobial activity.

Table 2, shown below, illustrates minimum inhibitory concentration (MIC) of silver acetate (AgAc) against laboratory and clinical isolates of P. aeruginosa and MRSA upon 2 h pre-incubation with 0 or 10 mM NAC.

TABLE 2

| | MIC of AgAc with | MIC of AgAc with |
|---|---|---|
| | 0 mM NAC pre- | 10 mM NAC pre- |
| Bacteria | incubation (μg/mL) | incubation (μg/mL) |
| PA O1 | 1 | 1 |
| PA M57-15 | 0.25 | 0.25 |
| PA HP3 | 1 | 1 |
| PA 14 | 1 | 1 |
| USA 300 | 2 | 2 |
| MRSA 0606 | 2 | 2 |
| MRSA 0638 | 2 | 2 |
| MRSA 0646 | 2 | 2 |

In accordance with an embodiment of the claimed invention, a silver/NAC combination presents a unique therapeutic strategy that can effectively eradicate bacterial infections without causing toxicity to eukaryotic cells. Cells incubated with silver demonstrate high levels of ROS, which causes disruption of the TCA cycle and reduction in ATP production, ultimately leading to cell death via apoptosis or necrosis. On the other hand, cells pre-incubated with NAC followed by silver do not demonstrate signs of oxidative stress, show a normal metabolic state, as well as ATP production, which translates to lower silver toxicity. Thus, the silver/NAC combination has tremendous potential as a therapeutic with potent antimicrobial activity with a large therapeutic window.

In view of the aforementioned, some embodiments of the present disclosure seek to provide delivery devices for localized delivery of antimicrobial, anti-inflammatory, and antioxidant agents.

Bacterial infections are one of the most common complications associated with several disease states including cystic fibrosis (CF), chronic obstructive pulmonary disorder (COPD), and chronic wounds such as diabetic foot ulcers, arterial and venous ulcers, as well as pressure ulcers. Multi-drug resistant Pseudomonas aeruginosa and Staphylococcus aureus are two of the most common pathogens responsible for these infections. In addition to the bacterial infection, these disease states are often in a pro-inflammatory state, further complicating the prognosis. Upon infection with bacteria, inflammation is further exacerbated, causing irreversible damage to a patient's lungs (for CF and COPD) or to the wound site (for chronic wounds).

Current standard-of-care (SoC) comprises of localized or systemic antimicrobial administration in conjunction with systemic administration of an anti-inflammatory agent such as non-steroidal anti-inflammatory drugs (NSAIDs). Ibuprofen is one of the most common NSAIDs employed to curb the runaway inflammation in these patients, however, the high risk of GI and renal toxicity hampers the use of ibuprofen. Localized use of ibuprofen can address these side-effects and provide anti-inflammatory activity of ibuprofen at the disease site. In addition, the infections and inflammation also gives rise to higher than normal levels of reactive oxygen species at the infection site, which are often deleterious. Current therapeutic devices can deliver either an anti-inflammatory agent or an antimicrobial agent locally, despite a need to deliver a combination of the two. Thus, there is an an urgent, unmet medical need to develop devices that can deliver a combination of an antimicrobial, anti-inflammatory, and antioxidant directly at the infection site.

In some embodiments, the present disclosure relates to two unique platforms that address the shortcomings detailed above by incorporating an anti-inflammatory agent, antimicrobial agent, and an antioxidant into one single device. The first platform comprises targeted nanoparticles or liposomes for systemic delivery or aerosolized for delivery to the lungs, while the second platform is an electrospun bandage to deliver the therapeutics directly to infected wounds. In addition, excipients, including but not limited to phospholipids, polyethylene glycol and its esters, and citric acid will be incorporated to provide favorable drug release, absorption, and stability.

In some embodiments, the delivery platforms can be nanoparticles. In some embodiments, the delivery platforms can be polymers, such as, for example, poly(caprolactone) and poly(lactic-co-glycolic acid). In some embodiments, the excepients can include, without limitation, to phospholipids, polyethylene glycol and its esters, citric acid and its salts, glucose, dextrose, lactose, sucrose, tocopherols, cysteine and its salts and esters, alkyl ammonium sulfonic acid betaine, ammonio methacrylate copolymers, arginine, aspartame, aspartic acid, boric acid, caffeine, lactic acid and its salts, carboxymethyl cellulose, carboxymethyl starch, cellulose and its esters, cholesterol, collagen, dextrin, dextran, dextrose, fructose, galactose, glucuronic acid, glutathione, lysine and its salts and esters, stearic acid and its salts, maleic acid and its salts and esters, maltodextrin, mannose, pectin, poly(lactic-co-glycolic) acid and its esters, polysaccharides, poly(vinyl alcohol), phosphoric acid and its salts, saccharin, alginates, boric acid and its salts, sodium chloride, sorbic acid and its esters, sorbitol, starch, succinic acid, sucralose, threonine, threacetin, valine, xylitol, hyaluronic acid and its esters and salts to impart stability.

In some embodiments, the liposomes can include, without limitation, to neutral and charged lipids, such as POPC, DHPE, DPPC. In some embodiments, the targeting ligands can include, without limitation, natural and synthetic glycans or glycolipids, such as GM1, GM2, GM3, fGM1, AGM1, AGM2, Gb3, Gb4, iGb3, GD1a, GD1b, GD2, GD3, LacCer, Gal-beta-Cer, GluCer, L8-L19, NA2, NGAG2, GD2, Curd-13, Curd-7, Curd-11, LacNac, Adi, GA2Di, GalNac-alpha, Forssman Di. The n-linked or o-linked glycans can be conjugated to PE lipids, forming synthetic glycolipids (or called neoglycolipids). The natural or synthetic glycolipids can be incorporated at various percentages (w/w) into the liposome, in order to target bacteria.

In some embodiments, the antioxidants can include, without limitation, ascorbic acid, melatonin, N-acetyl cysteine (NAC), polyphenols, anthocyanins, and flavanoids. In some embodiments, the anti-inflammatory agent can include, without limitation, NSAIDs such as, but not limited to ibuprofen, naproxen, indomethacin, and ketoprofen. In some embodiments, the antimicrobial agent can include, without limitation, silver, ceftazidime, amikacin, or minocycline.

In some embodiments, the delivery platforms can be in the form of fabricated bandages using electrospinning In some embodiments, fabrication of nanoparticle devices or micelles can be utilized for the delivery platforms using nanoprecipitation, emulsion, or electro-spraying techniques.

In some embodiments, the present disclosure relates to a combination of anti-inflammatory agents, antimicrobial agents, and antioxidants having been incorporated into one single device to achieve localized delivery at the infection site. These three components target unique conditions, chronic inflammation, bacterial infection, and ROS generation, that delay the healing process. The drug delivery devices described here, nanoparticles, micelles, and electrospun bandages, deliver a combination of antimicrobials, anti-inflammatory agents, and an antioxidants directly at the infection site to eradicate bacteria, attenuate chronic inflammation, and temper the free radicals. Further, these drug delivery devices provide controlled release of these agents directly at the infection site. These features help reduce the amount of therapeutic required to treat infection, as well as attenuate side-effects commonly associated with systemic administration.

Bacterial infections, particularly those involving MDR pathogens, present a grave threat to patients suffering from CF, COPD, and chronic wounds. Further, chronic inflammation exacerbates the condition of these patients and causes significant damage to the surrounding tissue. The delivery devices described herein can be utilized to address this urgent, unmet medical need. Moreover, in some embodiments, these devices have been designed using exclusively FDA-approved materials to fast track the approval process.

Currently, several bandages incorporated with an antimicrobial agent have been FDA approved and are currently used by clinicians as prophylactics or to eradicate bacterial infections in chronic wounds. A separate bandage approved for use provides localized delivery of ibuprofen at the wound site; however, it lacks potent antimicrobial activity required to treat bacterial infections. Moreover, since these are two separate bandages, they cannot be applied to the wound at the same time. Similarly, inhalable formulations of antimicrobials also exist to treat pulmonary bacterial infections, but these devices do not address the inflammation or ROS, mandating systemic administration of anti-inflammatory agents. The present disclosure seeks to remedy these aforementioned needs.

In some embodiments, the delivery systems of the present disclosure provide an antimicrobial, an antioxidant, and an anti-inflammatory agent, or agents, which are delivered directly to the infection site by a single device. In addition to the controlled release of antimicrobial directly at the infection site, these devices provide: (a) localized delivery of anti-inflammatory agents—this localized delivery of ibuprofen reduces GI and renal toxicity, commonly associated with long-term, high-dose NSAID intake; and (b) anti-oxidants to ameliorate free radicals generated at the infection site.

In some embodiments, the present disclosure utilizes to a combination of silver (Ag⁺ ion) as the antimicrobial agent and ibuprofen as the anti-inflammatory agent for the delivery agent.

Ibuprofen complements the antimicrobial efficacy of silver. A silver salt of ibuprofen (AgIBU) is formulated to provide a combination of antimicrobial and anti-inflammatory action from a single molecule. Antimicrobial activity of an AgIBU salt was compared to silver acetate (AgAc) using standard Clinical and Laboratory Standards Institute (CLSI) broth microdilution method. The minimum inhibitory and bactericidal concentration (MIC and MBC) against P. aeruginosa and methicillin-resistant Staphylococcus aureus (MRSA) demonstrate enhanced antimicrobial activity against majority of the tested isolates (8 out of 10 MRSA and 7 out of 9 P. aeruginosa isolates) upon treatment with AgIBU, as illustrated in Table 3 and Table 4, shown below.

Table 3, shown below, illustrates that silver ibuprofen (AgIBU) demonstrates superior antimicrobial activity (lower MIC) over silver acetate against 8 out of 10 tested MRSA strains.

TABLE 3

| | |
|---|---|
| Silver Acetate | Silver Ibuprofen |

| MRSA | MIC | MBC | MIC | MBC |
|---|---|---|---|---|
| 10 Strains | (μg/mL) | (μg/mL) | (μg/mL) | (μg/mL) |
| USA300 - TCH1516 | 16 | >32 | 4 | 32 |
| MRSA 0606 | 24 | >32 | 12 | >32 |
| MRSA 0608 | 24 | >32 | 4 | >32 |
| MRSA 0611 | 24 | >32 | 6 | >32 |
| MRSA 0631 | 24 | >32 | 8 | >32 |
| MRSA 0633 | 24 | >32 | 6 | >32 |
| MRSA 0638 | 24 | >32 | 6 | >32 |
| MRSA 0641 | 16 | >32 | 6 | >32 |
| MRSA 0646 | 16 | >32 | 16 | >32 |
| SAEH 06 | 8 | 12 | 16 | 32 |

Table 4, shown below, illustrates that silver ibuprofen demonstrates superior antimicrobial activity (lower MIC and MBC) over silver acetate against 7 out of 9 tested P. aeruginosa strains.

TABLE 4

| | |
|---|---|
| Silver Acetate | Silver Ibuprofen |

| P. aeruginosa | MIC | MBC | MIC | MBC |
|---|---|---|---|---|
| 9 Strains | (μg/mL) | (μg/mL) | (μg/mL) | (μg/mL) |
| PAM 57-15 | 4 | 6 | 4 | 4 |
| PA 05-31 | 4 | 6 | 2 | 4 |
| PA 05-40 | 4 | 6 | 2 | 4 |
| PA 05-45 | 4 | 6 | 2 | 4 |
| PA 05-51 | 4 | 6 | 4 | 4 |
| PA 05-54 | 4 | 6 | 4 | 4 |
| PA 05-57 | 6 | 6 | 4 | 4 |
| PAO1 | 4 | 4 | 4 | 4 |
| PA HP3 | 4 | 8 | 4 | 8 |

Antimicrobial activity of silver salt of ibuprofen against a panel of P. aeruginosa and MRSA. The preliminary screening that demonstrated superior activity of AgIBU compared with AgAc against P. aeruginosa and MRSA was then expanded to include a library of 45 MRSA clinical isolates and 31 P. aeruginosa isolates. The MIC90 and MBC90 which represent concentration required to inhibit and eradicate 90% of the tested isolates was then calculated using the individual MIC and MBC values. The AgIBU MIC90 and MBC90 values for P. aeruginosa were found to be 4 μg/mL. AgIBU acts as a bacteriostatic agent against MRSA with an MIC90 value of 12 μg/mL. Thus, the silver salt of ibuprofen demonstrates potent antimicrobial activity, which is superior to silver ion, and has an additional anti-inflammatory component in the form of ibuprofen.

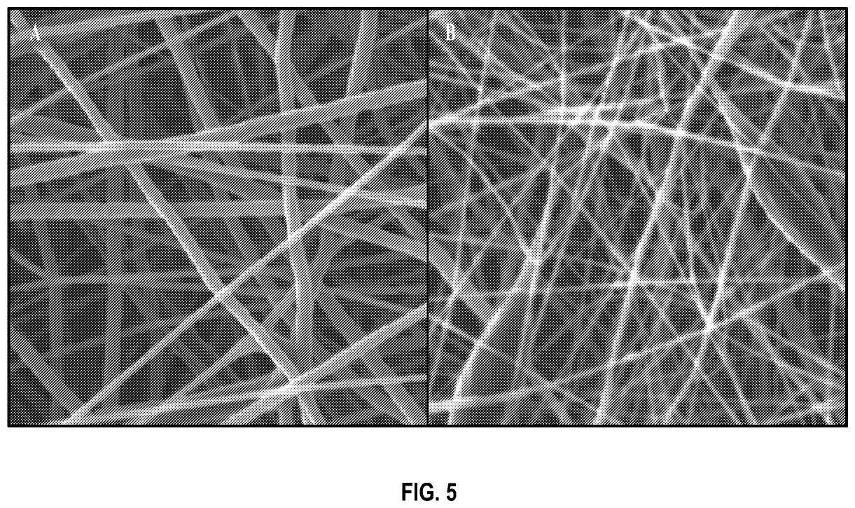

Fabrication of electro-spun scaffolds incorporated with silver salt of ibuprofen. Polycaprolactone, an FDA approved, biodegradable, polyester has been chosen as the core polymer for fabrication of bandages. These bandages are fabricated using electrospinning, a technique specifically chosen because of its ease, scalability and consistency. Further, electro-spun bandages mimic the extra-cellular matrix (ECM), which aids in the wound healing process. PCL and silver salt of ibuprofen were dissolved in organic solvents and electro-spun using standard technique to form the bandages. The scaffolds were then sputter coated with gold/palladium and imaged using a scanning electron microscope. FIG. 5 shows scanning electron micrographs of (a) drug-free and (b) AgIBU loaded poly(caprolactone) electro-spun scaffolds These scaffolds have been optimized for loading and can be incorporated with up to 20% AgIBU w/w. In addition, the inventors have also incorporated bandages with AgIBU as well as NAC to deliver antimicrobial, anti-inflammatory, and antioxidant at the infection site.

Lectin hetero-multivalency, binding to two or more different types of ligands, has been demonstrated to play a role in case of both LecA (a Pseudomonas aeruginosa adhesin) and Cholera Toxin subunit B (a Vibrio cholera toxin). In order to screen the ligand candidates that are involved in hetero-multivalent binding from large molecular libraries, turbidity-based emulsion agglutination (TEA) assays can be conducted in a high throughput format using standard laboratory instruments and reagents. The benefit of this assay is that it relies on the use of emulsions that can be formed using ultrasonication, minimizing the bottleneck of substrate surface functionalization. By measuring the change in turbidity, the lectin-induced aggregation rate of oil droplets could be quantified to determine the relative binding strength between different ligand combinations. The TEA results are consistent with prior binding results using a nanocube sensor. As such, the aforementioned TEA assay can serve as a high-throughput and customizable tool to screen the potential ligands involved in hetero-multivalent binding.

Furthermore, a single glycan-lectin interaction is often weak and semi-specific. Multiple binding domains in a single lectin can bind with multiple glycan molecules simultaneously, making it difficult for the classic “lock-and-key” model to explain these interactions. Hetero-multivalency influences LecA-glycolipid recognition Enhanced binding between P. aeruginosa and mixed glycolipid liposomes has been observed, and interestingly, strong ligands can activate weaker binding ligands leading to higher LecA binding capacity. Simulations identified the frequency of low-affinity ligand encounters with bound LecA and the bound LecA's retention of the low-affinity ligand as essential parameters for triggering hetero-multivalent binding, agreeing with experimental observations. The hetero-multivalency can alter lectin-binding properties, including avidities, capacities, and kinetics, and therefore, it likely occurs in various multivalent binding systems. Using hetero-multivalency concept, a new strategy to design high-affinity drug carriers for targeted drug delivery can be developed.

In view of the aforementioned hetero-multivalency, an aspect of the present disclosure further relates to direct targeting schemes for compositions of the drug delivery platforms presented herein. In some embodiments, adding glycan to compositions of the drug delivery platforms herein can provide for direct targeting of the antioxidant agent, anti-inflammatory agent, the antimicrobial agent, or combinations thereof. In some embodiments, the addition of glycan to liposomes facilitates in direct targeting of the antioxidant agent, anti-inflammatory agent, the antimicrobial agent, or combinations thereof, of the drug delivery platform to particular cells, proteins on a cell surface, tissue, infected areas, and the like. Furthermore, in some embodiments, the addition of glycan can increase the half-life of the drug delivery platform, for example, extending the half-life of a liposome. In some embodiments, the half-life can be extended in vivo or in vitro.

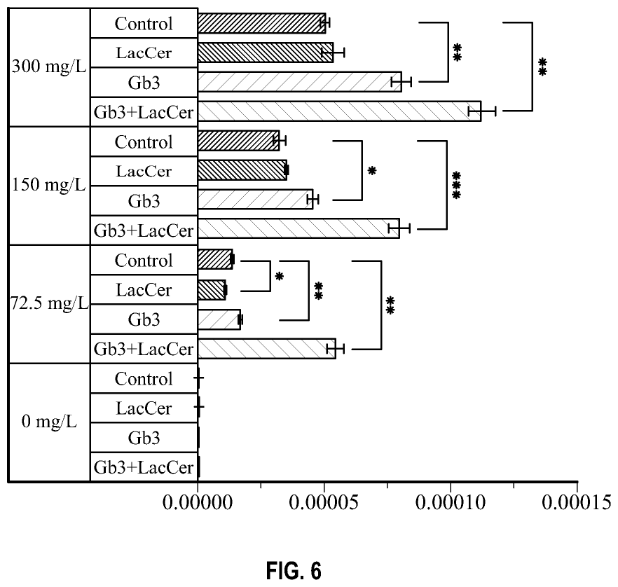

Hetero-multivalent targeting strategy increases the liposome attachment to PA. PA-specific targeted liposomes incorporated with either 10 mol % Gb3, 10 mol % LacCer, or an equi-molar combination of the two (5 mol % Gb3/5 mol % LacCer) were fabricated. Gb3 and LacCer were chosen as the targeting ligands since Gb3 is a strong ligand for LecA (a PA adhesion, as well as a linker in the PA biofilm matrix) and LacCer is a weak ligand for Type IV Pilus (T4P) of PA. Identical non-targeted formulations were fabricated and employed as controls. The bacteria (PAO1) were cultured for 48 h to establish stable biofilms. The targeting efficacy of liposomes was determined by measuring the retention of liposomes in the PA biofilms. The normalized fluorescence results from the liposomes bound to PA are shown in FIG. 6. The retention of the liposomes containing 10 mol % of LacCer, the weaker of the two ligands, was comparable to the control liposomes at all concentrations. The retention of 10 mol % of was slightly higher (5-30%) than the control liposomes. The Gb3/LacCer liposomes (5 mol %+5 mol %) demonstrated significantly higher retention over other liposomal formulations tested. Specifically, at the lowest concentration (72.5 mg/L), the Gb3/LacCer liposomes demonstrated over 4-fold higher attachment over Gb3-targeted, LacCer-targeted, and non-targeted liposomes. The formula of the control liposomes is similar to the commercially available clinical liposomes used to treat PA infections; thus, the inventors anticipate significant improvement in the clinical outcomes by incorporating eukaryotic cell molecules as targeting moieties. These results demonstrate the tremendous potential of mixed host cellular ligands to achieve liposomal targeting against PA in biofilms.

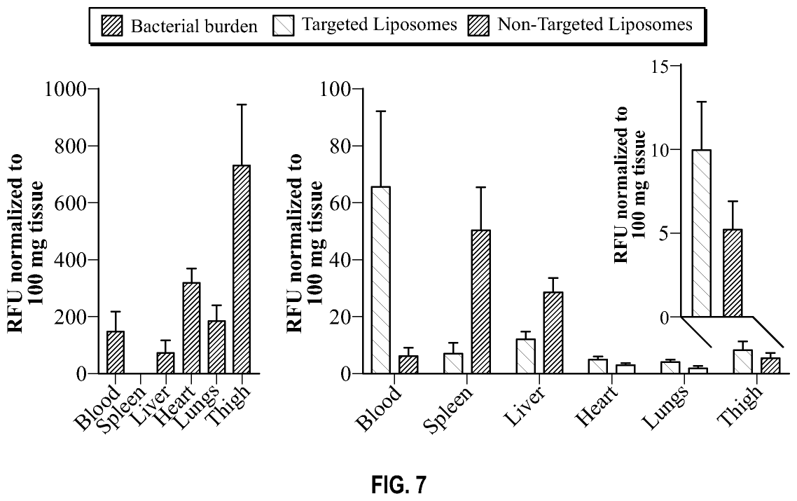

Targeted liposomes demonstrate co-localization with PA and increased residence time in vivo. Mice (CD-1, 5 per group), were injected with 1E8 CFU of Green Fluorescence Protein expressing PAO1 (GFP-PAO1) in the thigh muscle. One hour post-infection, mice were injected with 50 μL, 5 mg/mL targeted or non-targeted liposomes incorporated with Texas-red DHPE. Two hours later, the mice were anaesthetized, exsanguinated via cardiac blood draw, and blood, spleen, liver, heart, lungs, and thigh muscle harvested. The tissues were homogenized and fluorescence intensity from the bacteria and liposomes was measured using a microplate reader (FIG. 7). Targeted liposomes demonstrate a strong trend of localization with the bacteria in the blood, heart, lungs, and the thigh muscle after 2 h. Additionally, the targeted liposomes demonstrate an increased residence time in the blood as opposed to non-targeted liposomes that tend to localize in the spleen and the liver.

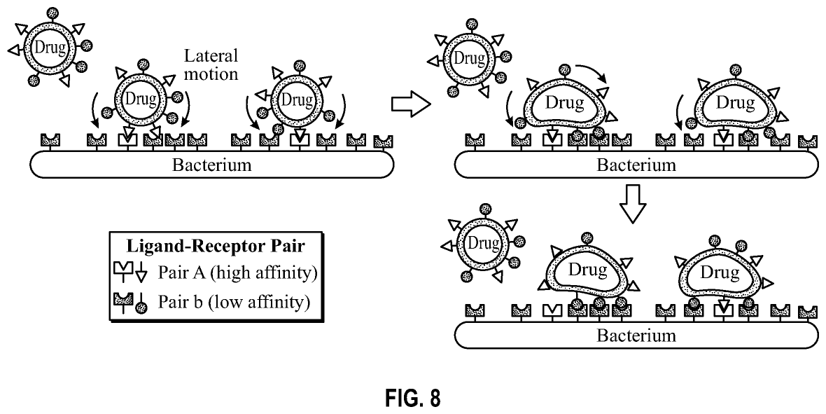

As shown in FIG. 8, the first binding between liposomes and bacteria occurs in 3D space. Liposomes diffuse from the solution phase to a bacterial surface and the first binding is likely initiated by a high affinity glycolipid on the liposome and receptor on bacterial surface. After the first attachment, the unbound glycolipids can diffuse two-dimensionally on the liposome surface, encounter their respective receptors and enable subsequent bindings. The reaction rate on a 2D surface is more than 100 times higher than the reaction rate in 3D space. Thus, low-affinity glycolipids can also contribute to subsequent binding events. The binding between any ligand-receptor pair is reversible. It is possible that even when the high-affinity receptor dissociates, the liposomal attachment to the bacteria remains stabilized by binding between multiple low-affinity ligand-receptor pairs only. The dissociated high-affinity receptor becomes available to bind with a new liposome and repeat the attachment process. This cycle in which high affinity ligand-receptor pairs only act as transient facilitators for liposomal attachment to bacteria to promote binding between low-affinity ligand-receptor pairs is an example of “ligand exchange.” Ultimately, hetero-multivalent binding between liposomes and bacteria results in higher retention of liposomes on the bacterial surface and attachment of a greater number of liposomes.

Although various embodiments of the present disclosure have been illustrated in the accompanying Drawings and described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1. A drug delivery platform comprising:
a polymeric material;
an excepient;
an antioxidant agent;
an anti-inflammatory agent; and
an antimicrobial agent, wherein the anti-inflammatory agent and the antimicrobial agent are formulated to provide a combination of antimicrobial and anti-inflammatory action from a single molecule.

2. (canceled)

3. The drug delivery platform of claim 1, wherein the polymeric material is selected from the group consisting of poly(caprolactone), poly(lactic-co-glycolic acid), cyclodextrin, and combinations thereof.

4. The drug delivery platform of claim 1, wherein the excepient is selected from the group consisting of phospholipids, polyethylene glycol and its esters, citric acid and its salts, glucose, dextrose, lactose, sucrose, tocopherols, cysteine and its salts and esters, alkyl ammonium sulfonic acid betaine, ammonio methacrylate copolymers, arginine, aspartame, aspartic acid, boric acid, caffeine, lactic acid and its salts, carboxymethyl cellulose, carboxymethyl starch, cellulose and its esters, cholesterol, collagen, dextrin, dextran, dextrose, fructose, galactose, glucuronic acid, glutathione, lysine and its salts and esters, stearic acid and its salts, maleic acid and its salts and esters, maltodextrin, mannose, pectin, poly(lactic-co-glycolic) acid and its esters, polysaccharides, poly(vinyl alcohol), phosphoric acid and its salts, saccharin, alginates, boric acid and its salts, sodium chloride, sorbic acid and its esters, sorbitol, starch, succinic acid, sucralose, threonine, threacetin, valine, xylitol, hyaluronic acid and its esters, salts to impart stability, and combinations thereof.

5. The drug delivery platform of claim 1, wherein the antioxidant agent is selected from the group consisting of ascorbic acid, melatonin, N-acetyl cysteine (NAC), polyphenols, anthocyanins, flavanoids, and combinations thereof.

6. The drug delivery platform of claim 1, wherein the anti-inflammatory agent is selected from the group consisting of an NSAID, ibuprofen, naproxen, indomethacin, ketoprofen, and combinations thereof.

7. The drug delivery platform of claim 1, wherein the antimicrobial agent is selected from the group consisting of silver, silver-containing compounds, sulfonamides, carbapenems, penicillins, diaminopyrimidines, quinolones, beta-lactam antibiotics, cephalosporins, tetracyclines, notribenzenes, aminoglycosides, macrolide antibiotics, polypeptide antibiotics, nitrofurans, nitroimidazoles, nicotinin acids, polyene antibiotics, imidazoles, glycopeptides, cyclic lipopeptides, glycylcyclines, oxazolidinones or other compounds, dapsone, paraminosalicyclic, sulfanilamide, sulfamethizole, sulfamethoxazole, sulfapyridine, trimethoprim, pyrimethamine, nalidixic acid, norfloxacin, ciproflaxin, cinoxacin, enoxacin, gatifloxacin, gemifloxacin, grepafloxacin, levofloxacin, lomefloxacin, moxifloxacin, ofloxacin, pefloxacin, sparfloxacin, trovafloxacin, amoxicillin, ampicillin, azlocillin, carbenicillin, cloxacillin, dicloxacillin, flucloxacillin, hetacillin, oxacillin, mezlocillin, penicillin G, penicillin V, piperacillin, cefacetrile, cefadroxil, cefalexin, cefaloglycin, cefalonium, cefaloridin, cefalotin, cefapirin, cefatrizine, cefazaflur, cefazedone, cefazolin, cefradine, cefroxadine, ceftezole, cefaclor, cefonicid, ceforanide, cefprozil, cefuroxime, cefuzonam, cefinetazole, cefoteta, cefoxitin, cefcapene, cefdaloxime, cefdinir, cefditoren, cefetamet, cefixime, cefinenoxime, cefodizime, cefoperazone, cefotaxime, cefotiam, cefpimizole, cefpiramide, cefpodoxime, cefteram, ceftibuten, ceftiofur, ceftiolen, ceftizoxime, ceftriaxone, cefoperazone, ceftazidime, cefepime, moxolactam, imipenem, ertapenem, meropenem, aztreonam, oxytetracycline, chlortetracycline, clomocycline, demeclocycline, tetracycline, doxycycline, lymecycline, meclocycline, methacycline, minocycline, rolitetracycline, chloramphenicol, amikacin, gentamicin, framycetin, kanamycin, neomicin, neomycin, netilmicin, streptomycin, tobramycin, azithromycin, clarithromycin, dirithromycin, erythromycin, roxithromycin, telithromycin, polymyxin-B, colistin, bacitracin, tyrothricin, notrifurantoin, furazolidone, metronidazole, timidazole, isoniazid, pyrazinamide, ethionamide, nystatin, amphotericin-B, hamycin, miconazole, clotrimazole, ketoconazole, fluconazole, rifampacin, lincomycin, clindamycin, spectinomycin, chloramphenicol, clindamycin, colistin, fosfomycin, loracarbef, nitrofurantoin, procain, spectinomycin, timidazole, ramoplanin, teicoplanin, and vancomycin, and combinations thereof.

8. The drug delivery platform of claim 1, wherein the drug delivery platform is in a form of a fabricated bandage formed by electrospinning or an aerosol.

9. The drug delivery platform of claim 1, wherein fabrication of nanoparticle devices or micelles are utilized for the drug delivery platform using nanoprecipitation, emulsion, or electro-spraying techniques.

10. The drug deliver platform of claim 1, comprising at least one of a lipid selected from natural eukaryotic cell lipids, as phospholipids, cholesterols, triglycerides, glycolipids, sphingolipids, and combinations thereof or a set of natural or synthetic glycans conjugated to lipids to bind to bacteria.

11. A drug delivery platform comprising:
a polymeric material;
an excepient;
an antioxidant agent; and
a combined anti-inflammatory and antimicrobial agent, wherein the combined anti-inflammatory and antimicrobial agent is a salt of an anti-inflammatory agent and an antimicrobial agent formulated to provide a combination of antimicrobial and anti-inflammatory action from a single molecule.

12. The drug delivery platform of claim 11, wherein the combined anti-inflammatory and antimicrobial agent is a silver salt of ibuprofen (AgIBU).

13. A method to treat inflammation, bacterial pathogens, multi-drug resistant (MDR) pathogens, MDR-Pseudomonas aeruginosa, or reactive oxygen species in a subject, the method comprising:
administering a drug delivery platform to a subject in need thereof; and
wherein the drug delivery platform comprises:
a polymeric material;
an excepient;
an antioxidant agent;
an anti-inflammatory agent; and
an antimicrobial agent, wherein the anti-inflammatory agent and antimicrobial agent are formulated to provide a combination of antimicrobial and anti-inflammatory action from a single molecule.

14. (canceled)

15. The method of claim 13, wherein the polymeric material is selected from the group consisting of poly(caprolactone), poly(lactic-co-glycolic acid), cyclodextrin, and combinations thereof.

16. The method of claim 13, wherein the excepient is selected from the group consisting of phospholipids, polyethylene glycol and its esters, citric acid and its salts, glucose, dextrose, lactose, sucrose, tocopherols, cysteine and its salts and esters, alkyl ammonium sulfonic acid betaine, ammonio methacrylate copolymers, arginine, aspartame, aspartic acid, boric acid, caffeine, lactic acid and its salts, carboxymethyl cellulose, carboxymethyl starch, cellulose and its esters, cholesterol, collagen, dextrin, dextran, dextrose, fructose, galactose, glucuronic acid, glutathione, lysine and its salts and esters, stearic acid and its salts, maleic acid and its salts and esters, maltodextrin, mannose, pectin, poly(lactic-co-glycolic) acid and its esters, polysaccharides, poly(vinyl alcohol), phosphoric acid and its salts, saccharin, alginates, boric acid and its salts, sodium chloride, sorbic acid and its esters, sorbitol, starch, succinic acid, sucralose, threonine, threacetin, valine, xylitol, hyaluronic acid and its esters, salts to impart stability, and combinations thereof.

17. The method of claim 13, wherein the antioxidant agent is selected from the group consisting of ascorbic acid, melatonin, N-acetyl cysteine (NAC), polyphenols, anthocyanins, flavanoids, and combinations thereof.

18. The method of claim 13, wherein the anti-inflammatory agent is selected from the group consisting of an NSAID, ibuprofen, naproxen, indomethacin, ketoprofen, and combinations thereof.

19. The method of claim 13, wherein the antimicrobial agent is selected from the group consisting of silver, silver-containing compounds, sulfonamides, carbapenems, diaminopyrimidines, quinolones, beta-lactam antibiotics, cephalosporins, tetracyclines, notribenzenes, aminoglycosides, macrolide antibiotics, polypeptide antibiotics, nitrofurans, nitroimidazoles, nicotinin acids, polyene antibiotics, imidazoles, glycopeptides, cyclic lipopeptides, glycylcyclines, and oxazolidinones or other compounds, dapsone, paraminosalicyclic, sulfanilamide, sulfamethizole, sulfamethoxazole, sulfapyridine, trimethoprim, pyrimethamine, nalidixic acid, norfloxacin, ciproflaxin, cinoxacin, enoxacin, gatifloxacin, gemifloxacin, grepafloxacin, levofloxacin, lomefloxacin, moxifloxacin, ofloxacin, pefloxacin, sparfloxacin, trovafloxacin, amoxicillin, ampicillin, carbenicillin, cloxacillin, dicloxacillin, flucloxacillin, betaciilin, oxacillin, mezlocillin, G, penicillin V, piperacillin, cefacetrile, cefadroxil, cefalexin, cefaloglycin, cefalonium, cefaloridin, cefalotin, cefapirin, cefatrizine, cefazaflur, cefazedone, cefazolin, cefradine, cefroxadine, ceftezole, cefaclor, cefonicid, ceforanide, cefprozil, cefuroxime, cefuzonam, cefinetazole, cefoteta., cefoxitin, cefcapene, cefdaioxime, cefdinir, cefditoren, cefetamet, cefixime, cefinenoxime, cefodizime, cefoperazone, cefotaxime, cefotiam, cefpimizole, cefpiramide, cefpodoxime, cefteram, ceftibuten, ceftiofur, ceftiolen, ceftizoxirne, ceftriaxone, cefoperazone, ceftazidime, cefepime, moxolactam, imipenem, ertapenem, meropenem, aztreonam, oxytetracycline, chlortetracycline, clomocycline, demeclocycline, tetracycline, doxycycline, lymecycline, meclocycline, methacycline minocycline, rolitetracycline, chlorarnphenicol, amikacin, gentamicin, framycetin, kanamycin, neomicin, neomycin, netilmicin, streptomycin, tobramycin, azithromycin, clarithromycin, dirithromycin, erythromycin, roxithromycin, telithromycin, polymyxin-B, bacitracin, tyrothricin, notrifurantoin, furazolidone, metronidazole, tirnidazole, isoniazid, pyrazinamide, ethionamide, nystatin, amphotericin-B, hamycin, miconazole, clotrimazole, ketoconazole, fluconazole, rifampacin, lincomycin, clindamycin, spectinomycin, chloramphenicol, clindamycin, colistin, fosfomycin, loracarbef, nitrofurantoin, procain, spectinonlycin, timidazole, ramoplanin, teicoplanin, and vancomycin, and combinations thereof.

20. The method of claim 13, wherein the drug delivery platform is in a form of a fabricated bandage formed by electrospinning or an aerosol.

21. The method of claim 13, wherein fabrication of nanoparticle devices or micelles are utilized for the drug delivery platform using nanoprecipitation, emulsion, or electro-spraying techniques.

22. The method of claim 13, wherein the drug delivery platform further comprises at least one of a lipid selected from natural eukaryotic cell lipids, such as phospholipids, cholesterols, triglycerides, glycolipids, sphingolipids, and combinations thereof, or a set of natural or synthetic glycans conjugated to lipids to bind to bacteria.

23-30. (canceled)
